(19) United States
(12) Patent Application Publication
(10) Pub. No.: US 2026/0090719 A1
(43) Pub. Date: Apr. 2, 2026

(54) METHOD AND SYSTEM FOR PROCESSING RETINAL IMAGES

(71) Applicant: OPTINA DIAGNOSTICS, INC., Montreal (CA)

(72) Inventors: Claudia CHEVREFILS, Montreal (CA); Gabriel DIGNARD, Montreal (CA); Wei YOU, Montreal (CA); Jean-Philippe SYLVESTRE, Montreal (CA)

(21) Appl. No.: 19/344,847

(22) Filed: Sep. 30, 2025

Related U.S. Application Data
(63) Continuation of application No. PCT/IB2024/053050, filed on Mar. 28, 2024.
(60) Provisional application No. 63/493,422, filed on Mar. 31, 2023.

(51) Int. Cl. A61B 3/15; A61B 3/00; A61B 3/12

(52) U.S. Cl. CPC A61B 3/152; A61B 3/0025; A61B 3/0033; A61B 3/0041; A61B 3/12

(57) ABSTRACT

A computer-implemented method of processing retinal images comprises accessing a multispectral image of a retina, the multispectral image comprising a first image of the retina associated with a first wavelength and a second image of the retina associated with a second wavelength, the second wavelength being distinct from the first wavelength, determining a first extent of a first artefact associated with the first image, determining a second extent of the first artefact or of a second artefact associated with the second image, computing a quality index based on the first extent of the first artefact and the second extent of the first or second artefact; and determining, based on the quality index, whether the multispectral image is suitable for training a machine-learning algorithm (MLA).

### CROSS-REFERENCE

The present patent application is a continuation of PCT Patent Application No. PCT/IB2024/053050, filed Mar. 28, 2024, and entitled “METHOD AND SYSTEM FOR PROCESSING RETINAL IMAGES”, which claims priority from U.S. provisional patent application Ser. No. 63/493,422, filed on Mar. 31, 2023, the disclosure of which is incorporated by reference herein in its entirety.

### FIELD

The present technology relates to systems and methods of medical imaging. In particular, the systems and methods for processing retinal images.

### BACKGROUND

Training of artificial intelligence (AI) systems with low number of data can be optimal when high quality data is available. In the context of medical imaging, to ensure that high quality data is used during training, a technician needs to take multiple acquisitions of images from the same subjects (i.e. patients) until a few good quality images is believed to have been acquired.

A technique for imaging a biological tissue is described in U.S. Pat. No. 10,964,036 to Jean Philippe Sylvestre et al., issued on Mar. 30, 2021, the disclosure of which is incorporated by reference herein in its entirety. Sylvestre teaches obtaining images of the biological tissue over multiple wavelengths. Such multispectral images, acquired from subjects in a clinical setup, may be used to train AI systems, inasmuch as they are of sufficient quality.

Regardless of the acquisition technique used, the images are conventionally inspected by a highly skilled AI specialist who selects those that are of sufficient quality for training of the AI system. This inspection is performed by an AI specialist after patient imaging session is completed. When the images are not good enough for AI training, the patient needs to be rejected from the database. Having a real time tool to identify if the images are of good quality would allow the technician to re-image the patient on the spot and eliminate the patient rejection.

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches.

### SUMMARY

Embodiments of the present technology have been developed based on developers' appreciation of shortcomings associated with the prior art.

In particular, such shortcomings may comprise the important technical skill level and related high cost that are required to acquire and evaluate images of sufficient quality for, in sufficient numbers, training of artificial intelligence systems.

In one aspect, various implementations of the present technology provide a computer-implemented method of processing retinal images, the method comprising:
accessing a multispectral image of a retina, the multispectral image comprising a first image of the retina associated with a first wavelength and a second image of the retina associated with a second wavelength, the second wavelength being distinct from the first wavelength;
determining a first extent of a first artefact associated with the first image;
determining a second extent of the first artefact or of a second artefact associated with the second image;
computing a quality index based on the first extent of the first artefact and the second extent of the first or second artefact; and
determining, based on the quality index, whether the multispectral image is suitable for training a machine-learning algorithm (MLA).

In some implementations of the present technology, accessing multispectral image comprises accessing a readable-computer medium comprising one or more files having been generated by a multispectral retinal camera.

In some implementations of the present technology, determining, based on the quality index, whether the multispectral image is suitable for training the MLA comprises comparing the quality index with a quality threshold; and if determination is made that the quality index does not at least fulfill the quality threshold, then determine that the multispectral image is not suitable for training the MLA.

In some implementations of the present technology, the method comprises the step of, if the multispectral image is deemed not to be suitable for the training of the MLA, generating instructions to acquire a replacement multispectral image of the retina.

In some implementations of the present technology, the generating instructions to acquire the replacement multispectral image of the retina iterates until a new quality index for the replacement multispectral image at least fulfills the quality threshold, the quality threshold establishing that the replacement multispectral image is suitable for training the MLA.

In some implementations of the present technology, the instructions comprise human-readable instructions to guide an operator with the acquisition of the at least one replacement multispectral image of the retina.

In some implementations of the present technology, the quality threshold is associated with the first and/or the second artefacts.

In some implementations of the present technology, the quality threshold is associated with a given biomarker.

In some implementations of the present technology, the quality threshold is one of a first threshold usable for pretraining the MLA and a second threshold usable for further training of the MLA, the second threshold being stricter than the first threshold.

In some implementations of the present technology, the further training of the MLA is used for fine tuning of the MLA on one or more specific tasks.

In some implementations of the present technology, the method comprises displaying a pictogram, the pictogram being associated with a color-code, the color-code being determined based on the quality index.

In some implementations of the present technology, multispectral image of the retina defines a hyperspectral cube, the hyperspectral cube being acquired by a hyperspectral camera.

In some implementations of the present technology, the first and second wavelengths are adjacent within the multispectral image.

In some implementations of the present technology, determining the first extent of the first artefact and determining the second extent of the first or second artefact are computed by executing at least one routine selected from a blur detection routine; a ghost detection routine; a blinks detection routine; an optical nerve head (ONH) position detection routine; a defocus detection routine; and a combination thereof.

In some implementations of the present technology, computing the quality index comprises initiating the quality index with a first numerical value; and modifying the quality index to obtain a second numerical value of the quality index, the modifying of the quality index being based a result of executing at least one of the routines.

In some implementations of the present technology, computing the quality index further comprises executing a plurality of the routines in an order selected according to a type of biomarker being sought in the multispectral image; and associating distinct weights to each of the plurality of the routines, the weights being selected according to the type of biomarker being sought in the multispectral image.

In some implementations of the present technology, the quality index is associated with a gradeability score selected from gradable, moderate, moderate_low and ungradable.

In some implementations of the present technology, the method comprises training the MLA based on the multispectral image of the retina, the trained MLA comprising a classification model being configured for detecting specific biomarkers and/or predicting medical conditions.

In some implementations of the present technology, the method comprises repeating the accessing of successive multispectral images and the determining whether the successive multispectral images are suitable for training the MLA until a predetermined number of suitable multispectral images have been acquired.

In some implementations of the present technology, the method comprises repeating the accessing of successive multispectral images and the determining whether the successive multispectral images are suitable for training the MLA until a combination of the quality indexes for the successive multispectral images fulfills a predetermined combined quality threshold.

In another aspect, various implementations of the present technology provide a computer-implemented method of processing medical images, the method comprising:
accessing a multispectral image of a biological tissue, the multispectral image comprising a first image of the biological tissue associated with a first wavelength and a second image of the retina associated with a second wavelength, distinct from the first wavelength;
determining a first extent of a first artefact associated with the first image;
determining a second extent of the first artefact or of a second artefact associated with the second image;
computing a quality index based on the first extent of the first artefact and the second extent of the first or second artefact; and
determining, based on the quality index, that the multispectral image is suitable for detection of medical conditions.

In a further aspect, various implementations of the present technology provide a non-transitory computer-readable medium storing computer-readable instructions that, when executed by a computer, cause the computer to perform the method of processing retinal images.

In yet another aspect, various implementations of the present technology provide a computer-implemented system configured to perform the method of processing retinal images.

In the context of the present specification, unless expressly provided otherwise, a computer system may refer, but is not limited to, an “electronic device”, an “operation system”, a “system”, a “computer-based system”, a “controller unit”, a “monitoring device”, a “control device” and/or any combination thereof appropriate to the relevant task at hand.

In the context of the present specification, unless expressly provided otherwise, the expression “computer-readable medium” and “memory” are intended to include media of any nature and kind whatsoever, non-limiting examples of which include RAM, ROM, disks (CD-ROMs, DVDs, floppy disks, hard disk drives, etc.), USB keys, flash memory cards, solid state-drives, and tape drives. Still in the context of the present specification, “a” computer-readable medium and “the” computer-readable medium should not be construed as being the same computer-readable medium. To the contrary, and whenever appropriate, “a” computer-readable medium and “the” computer-readable medium may also be construed as a first computer-readable medium and a second computer-readable medium.

In the context of the present specification, unless expressly provided otherwise, the words “first”, “second”, “third”, etc. have been used as adjectives only for the purpose of allowing for distinction between the nouns that they modify from one another, and not for the purpose of describing any particular relationship between those nouns.

Implementations of the present technology each have at least one of the above-mentioned objects and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects, and advantages of implementations of the present technology will become apparent from the following description, the accompanying drawings, and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 is an illustration of a clinical setup for acquiring images of the retina of a subject in accordance with an implementation of the present technology;

FIG. 2 is a schematic representation of a retinal image acquisition technique in accordance with an implementation of the present technology;

FIG. 3 is a schematic representation of an image acquisition, evaluation, and reacquisition process in accordance with an implementation of the present technology;

FIGS. 4a-4c are a sequence diagram showing operations of a computer-implemented method of processing retinal images in accordance with an implementation of the present technology; and

FIG. 5 is a block diagram showing operations of a quality index calculation method in accordance with an implementation of the present technology;

FIG. 6 is a block diagram of a computer-implemented system for processing retinal images in accordance with an implementation of the present technology.

It should also be noted that, unless otherwise explicitly specified herein, the drawings are not to scale.

### DETAILED DESCRIPTION

The examples and conditional language recited herein are principally intended to aid the reader in understanding the principles of the present technology and not to limit its scope to such specifically recited examples and conditions. It will be appreciated that those skilled in the art may devise various arrangements that, although not explicitly described or shown herein, nonetheless embody the principles of the present technology.

Furthermore, as an aid to understanding, the following description may describe relatively simplified implementations of the present technology. As persons skilled in the art would understand, various implementations of the present technology may be of a greater complexity.

In some cases, what are believed to be helpful examples of modifications to the present technology may also be set forth. This is done merely as an aid to understanding, and, again, not to define the scope or set forth the bounds of the present technology. These modifications are not an exhaustive list, and a person skilled in the art may make other modifications while nonetheless remaining within the scope of the present technology. Further, where no examples of modifications have been set forth, it should not be interpreted that no modifications are possible and/or that what is described is the sole manner of implementing that element of the present technology.

Moreover, all statements herein reciting principles, aspects, and implementations of the present technology, as well as specific examples thereof, are intended to encompass both structural and functional equivalents thereof, whether they are currently known or developed in the future. Thus, for example, it will be appreciated by those skilled in the art that any block diagrams herein represent conceptual views of illustrative circuitry embodying the principles of the present technology. Similarly, it will be appreciated that any flowcharts, flow diagrams, state transition diagrams, pseudo-code, and the like represent various processes that may be substantially represented in non-transitory computer-readable media and so executed by a computer or processor, whether or not such computer or processor is explicitly shown.

The functions of the various elements shown in the figures, including any functional block labeled as a “processor”, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. In some implementations of the present technology, the processor may be a general-purpose processor, such as a central processing unit (CPU) or a processor dedicated to a specific purpose, such as a digital signal processor (DSP). Moreover, explicit use of the term a “processor” should not be construed to refer exclusively to hardware capable of executing software, and may implicitly include, without limitation, application specific integrated circuit (ASIC), field programmable gate array (FPGA), read-only memory (ROM) for storing software, random access memory (RAM), and non-volatile storage. Other hardware, conventional and/or custom, may also be included.

Software modules, or simply modules which are implied to be software, may be represented herein as any combination of flowchart elements or other elements indicating performance of process steps and/or textual description. Such modules may be executed by hardware that is expressly or implicitly shown. Moreover, it should be understood that module may include for example, but without being limitative, computer program logic, computer program instructions, software, stack, firmware, hardware circuitry or a combination thereof which provides the required capabilities.

In an aspect, the present technology determines whether a multispectral image of a retina is suitable for training a machine-learning algorithm (MLA). The multispectral image comprises a plurality of images, or frames, acquired at a corresponding plurality of wavelengths. A first image of the retina is associated with a first wavelength and a second image of the retina is associated with a second wavelength, the second wavelength being distinct from the first wavelength. A first extent of a first artefact associated with the first image and a second extent of a second artefact associated with the second image are determined. In a non-limiting implementation, detection of some specific artefacts is better obtained using consecutive frames obtained in adjacent wavelengths. A quality index is computed based on the first extent of the first artefact and the second extent of the second artefact. The suitability of the multispectral image of the retina for training the MLA is determined based on the quality index.

With these fundamentals in place, we will now consider some non-limiting examples to illustrate various implementations of aspects of the present technology.

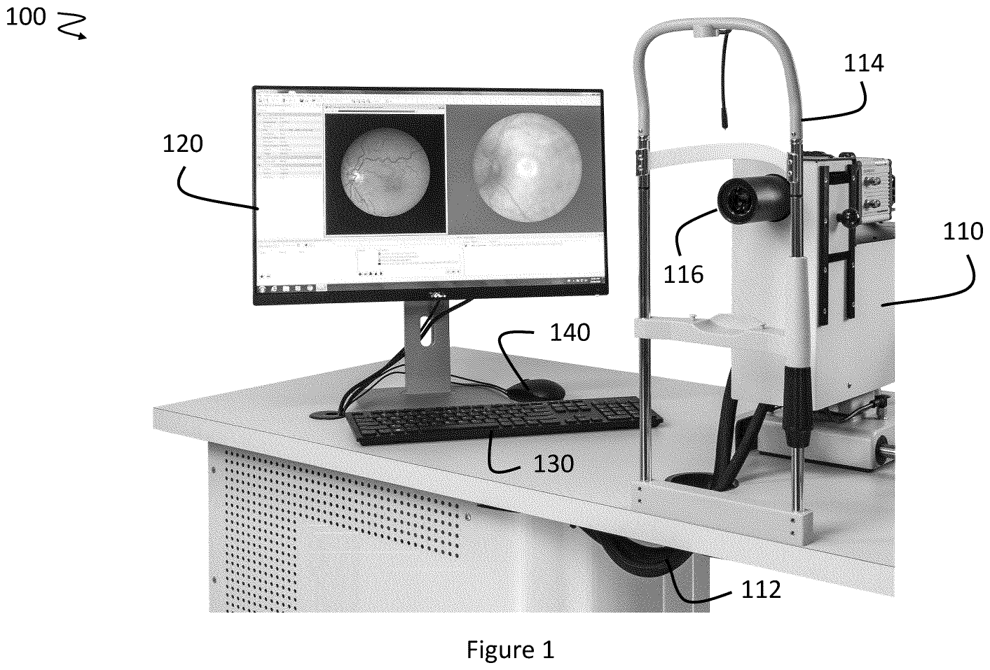

FIG. 1 is an illustration of a clinical setup 100 for acquiring images of the retina of a subject. The clinical setup comprises a multispectral retinal camera 110. The multispectral retinal camera 110 is connected via cables 112 to a computer (not shown) that, in turn, is connected to a computer monitor 120 and one or more human-machine interface devices, for example a keyboard 130 and a computer mouse 140. The multispectral retinal camera 110 includes an adjustable headrest 114 that allows placing an eye of the subject in front of imaging optics 116 for illuminating the retina and for capturing a multispectral image of the retina. The multispectral image is converted in numerical form by the multispectral retinal camera 110 and transmitted to the computer for processing.

The multispectral retinal camera 110 is configured to acquire images of the retina of the patient containing at least two frames captured over at least two distinct wavelengths. In the context of the present technology, a “wavelength” is to be understood as being defined in a narrow range. The multispectral image may include more than two distinct frames and wavelengths. In an implementation, the multispectral image of the retina may be a hyperspectral image acquired using a hyperspectral camera. For example and without limitation, the hyperspectral image may form a hyperspectral cube comprising up to 92 images, or frames, acquired at 92 respective wavelengths within visible and/or infrared spectra, each of wavelengths being defined within a 5 nanometers (nm) range, with no significant overlap between these ranges in order to minimize redundancy of information between the various images of the hyperspectral cube. It is contemplated that the present technology may be employed using multispectral images having other numbers of images with their respective wavelengths, including when some spectral overlap is present between wavelength ranges of each distinct images.

The multispectral image of the retina may be displayed on the computer monitor 120, along with a set of parameters used for capturing the multispectral image and/or with results of a computer analysis of the multispectral image. The keyboard 130 and the computer mouse 140 may be used by an operator to configure parameters used for capturing the multispectral image, for operating the multispectral retinal camera 110, for controlling storing of the multispectral image in a memory or database (not shown), and/or for transmission of the multispectral image to another computer, server, and the like. Processing of the multispectral image by the computer will be described hereinbelow.

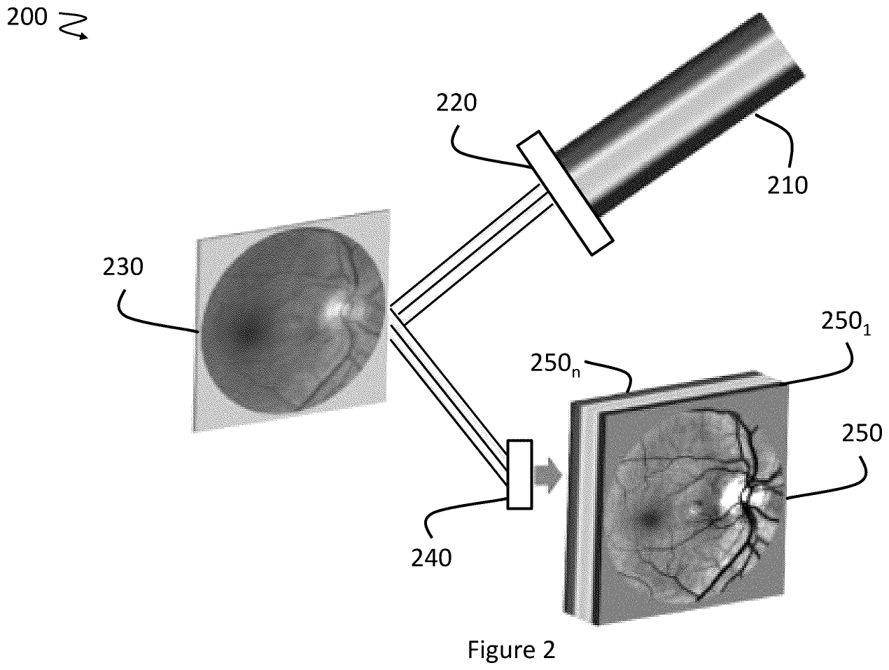

FIG. 2 is a schematic representation of a retinal image acquisition technique 200. Light 210 emitted at a plurality of distinct wavelengths is directed by a first optical element 220, part of the imaging optics 116 (FIG. 1), toward the eye of the subject, reaching the retina 230. Light reflected by the retina 230 is captured by a sensor 240, which is also part of the imaging optics 116. Other components of the imaging optics 116, for example lenses and/or mirrors, are not shown in order to simplify the illustration.

Each distinct wavelength of the light 210 may be emitted sequentially, so that the retina is illuminated at a single and narrow wavelength at once. The sequence may be executed rapidly, for example illuminating the retina 230 at 92 distinct wavelengths in less than one second, in order to reduce chances of the patient blinking or moving while the distinct images are captured. It is contemplated that the retina 230 may be illuminated by light over a broad spectrum, the sensor 240 selecting light at each single wavelength over a similarly brief period. It is also contemplated that the separation of light between the distinct wavelengths may be performed by digital post-processing after acquisition of the retinal image over a broad spectrum, the separation of light being performed either by the multispectral retinal camera 110 or by the computer. The present technology is therefore not limited by the manner in which the retinal image is acquired or by the manner in which the distinct wavelengths of the retinal image are separated.

The multispectral retinal camera 110 (or the computer) produces a “multispectral cube 250” (which may also be called “hyperspectral cube” if the multispectral retinal camera 110 is a hyperspectral camera), the multispectral cube 250 comprising a plurality of two-dimensional images, each image being acquired at a respective wavelength 250₁ . . . 250ₙ. Although the term “cube” is used herein, the two-dimensional images are not necessarily square. The two-dimensional images are defined in numbers of pixels in each dimension and the third dimension of the cube is defined in numbers of wavelengths.

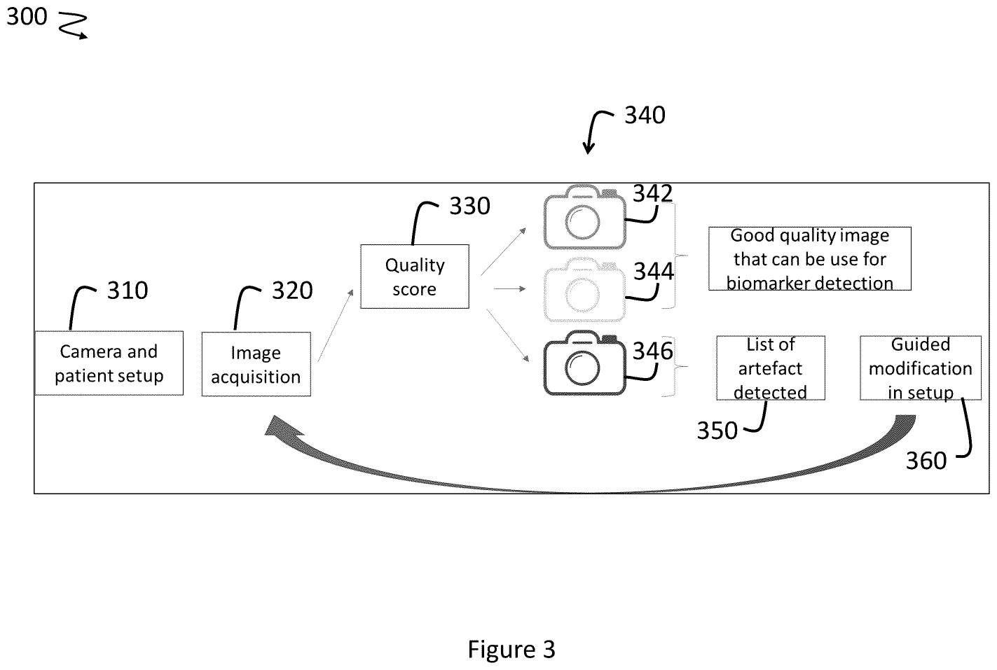

FIG. 3 is a schematic representation of an image acquisition, evaluation, and reacquisition method 300. The multispectral retinal camera 110 is set up by the operator and the subject (i.e. patient) is installed in view of the multispectral camera 110 at operation 310. The multispectral image of the retina is acquired at operation 320. A quality index, i.e. a score, is computed by the computer at operation 330. A pictogram 342, 344 or 346 is shown on the computer monitor 120 at operation 340, a color and/or shape of the pictogram being selected by the computer according to a value of the quality index. If the quality index has a value that at least fulfills a strict threshold defined for a given biomarker being sought in the multispectral image, the multispectral image is considered “gradable” and the pictogram 342 is displayed, being for example in green. For the given biomarker, two more thresholds may be used to evaluate, based on the quality index, that the multispectral image is considered “moderate” or “moderate_low”; in both cases, the pictogram 344 is displayed, being for example in yellow. The yellow pictogram 344 may advise an operator that, although the multispectral image is usable, some adjustment of the imaging components illustrated on FIGS. 1 and 2 may be appropriate. The threshold used for considering that the multispectral image is considered “moderate” is stricter than the threshold used for considering that the multispectral image is considered “moderate_low”. Likewise, the threshold used for considering that the multispectral image is considered “gradable” is stricter than the threshold used for considering that the multispectral image is considered “moderate”. Whether the multispectral image is considered gradable, moderate, or moderate_low, it is considered as a good quality image that can be used for detecting a given biomarker in the retina of the subject, and/or used for training the MLA. If the multispectral image is considered ungradable, the computer monitor 120 display the pictogram 346, being for example in red. An alternative implementation may assign the yellow pictogram 344 solely to those multispectral images having a moderate grade indication, so that multispectral images that are found to be graded as moderate_low are rejected.

Distinct sets of thresholds may be defined for distinct biomarkers being sought in the multispectral image.

Upon displaying the pictogram 346, the computer monitor 120 also displays, at operation 350, a list of one or more artefacts of the retina having been identified based on an analysis of the multispectral image. The computer monitor 120 further displays, at operation 360, instructions intended to guide the operator in modifying the setup of the multispectral camera 110 and/or of the subject in a repetition of the sequence 300 that may start again at operation 320, in view of acquiring a replacement multispectral image of the retina of the subject.

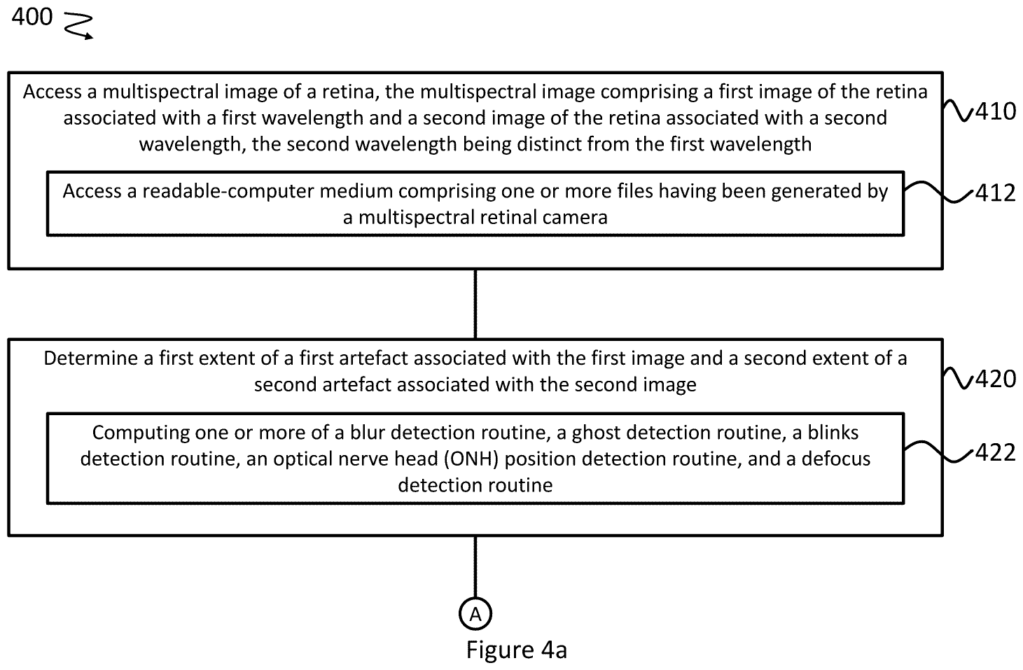

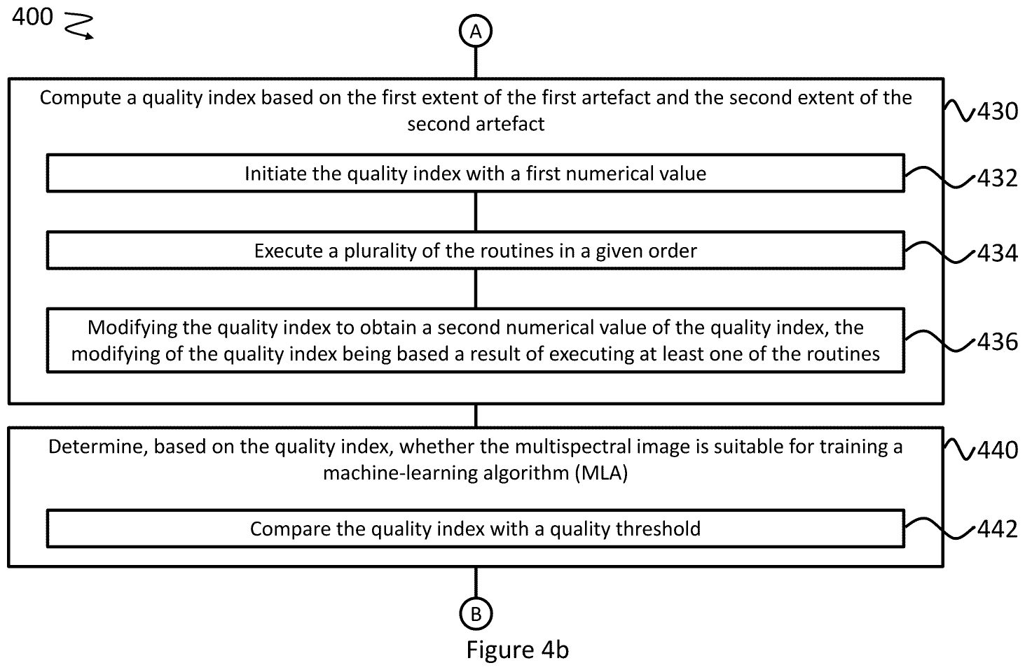

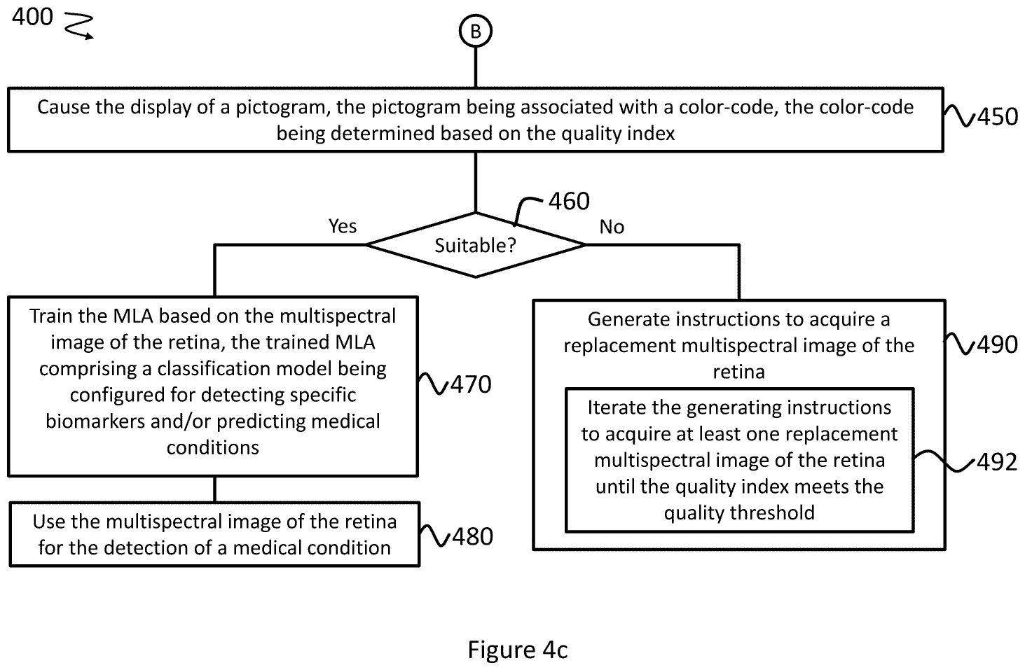

In more details, FIGS. 4a-4c are a sequence diagram showing operations of a computer-implemented method of processing retinal images. On FIGS. 4a-4c, a sequence 400 comprises a plurality of operations, some of which may be executed in variable order, some of the operations possibly being executed concurrently, some of the operations being optional. As shown on FIG. 4a, the sequence 400 is initiated at operation 410 by accessing, at a computer, a multispectral image of a retina. The multispectral image comprises a first image of the retina associated with a first wavelength and a second image of the retina associated with a second wavelength, the second wavelength being distinct from the first wavelength. Operation 410 may include sub-operation 412, in which a readable-computer medium comprising one or more files having been generated by the multispectral retinal camera 110 is accessed by the computer.

At operation 420, the computer determines a first extent of a first artefact associated with the first image and a second extent of a second artefact associated with the second image. In an implementation, the first artefact and the second artefact may be a same artefact for which the first extend and the second extend are determined at the first wavelength and at the second wavelength, respectively. In the same or in another implementation, the first and second images may comprise adjacent frames captured on adjacent wavelengths within the multispectral image. Operation 420 may comprise sub-operation 422, in which one or more of a blur detection routine, a ghost detection routine, a blinks detection routine, an optical nerve head (ONH) position detection routine, and a defocus detection routine is computed.

Continuing on FIG. 4b, the computer computes a quality index at operation 430. The quality index is based on the first extent of the first artefact and the second extent of the second artefact. The quality index may for example be associated with a grade selected from gradable, moderate, moderate_low and ungradable, depending on values of the quality index and on the various quality thresholds used for the given biomarker sought in the multispectral image. Operation 430 may comprise sub-operations 432, 434 and 436.

The quality index computation is initiated at sub-operation 432 with a first numerical value. At sub-operation 434, a plurality of the routines are executed in an order that may depend on the type of biomarker being sought. To this end, distinct weights may be associated to each of the plurality of the routines so that a modifying numerical value for each routine may be determined according to their respective weights, the weights being selected according to the type of biomarker being sought in the multispectral image. Execution of the routines may thus provide one or more modifying numerical values that are used, at sub-operation 436, to modify the first numerical value to obtain the second numerical value for the quality index. Starting from an “ideal” first numerical value for the quality index, the routines may reveal that the multispectral image is less than ideal when the routines detect that the multispectral image is blurred, ghosted, the ONH is not properly located by the image, focus of the multispectral image is lacking, or the subject has blinked their eyes at the time of multispectral image capture.

Various equivalent calculation techniques may be used. For example and without limitation, the quality index may ideally be equal to zero, and the modifying numerical values resulting from the execution of the routines may be positive integer or positive fractional values that are added to arrive at a global score for the quality index. In such case, a quality threshold is fulfilled when the global score for the quality index does not exceed the quality threshold.

In another non-limiting example, the quality index may ideally be a relatively high numerical value, so the first numerical value may be set to one (or 100%). The modifying numerical values may represent fractions of one (or some percentage values) that are subtracted from the first numerical value to obtain, as a global score, the second numerical value of the quality index, generally a positive value equal to or less than one (or 100%). In such case, a quality threshold is fulfilled when the global score for the quality index at least meets or exceeds the quality threshold. The manner in which the quality index is calculated does not limit the scope of the present disclosure.

Table I shows a possible relationship between the computed quality index and gradeability scores:

TABLE I
Gradeability Scores

| | |
|---|---|
| Gradable | 90% and up |
| Moderate | Between 80% to 90% |
| Moderate_low | Between 60% to 80% |
| Ungradable | Below 60% |

The quality index values presented in Table I are non-limiting, and other thresholds may be used between the various grade levels. In particular, distinct sets of thresholds may be defined depending on the type of biomarker being sought in the multispectral image.

At operation 440, the computer determines, based on the quality index (i.e. on the second numerical value of the quality index), whether the multispectral image is suitable for training a machine-learning algorithm (MLA). This operation 440 may include sub-operation 442, in which the computer compares the quality index value obtained at sub-operation 436 with a quality threshold (which may be specifically defined for a biomarker being sought in the multispectral image) and determines that the multispectral image is suitable for training the MLA if the quality index at least fulfills the quality threshold. Depending on the selected method for calculating the numerical values, the computer may determine that the multispectral image is suitable for training the MLA when the quality index is greater than or equal to the quality threshold, or strictly greater than the quality threshold, or lower than or equal to the quality threshold, or strictly lower than the quality threshold. In an implementation, a relatively lenient quality threshold may be applied, so that multispectral images of modest quality may still be used for pretraining of the MLA. A stricter quality threshold may be applied to select better multispectral images for further training of the MLA. In particular, the MLA may be trained further for fine tuning and optimization of the MLA on one or more specific tasks. Fine tuning on specific tasks may include, for example and without limitation, automatic segmentation of anatomical structures like vessels, Optical Nerve Head (ONH) or various lesion like Geographic Atrophy (GA), Pigment Anomaly (PA), drusen, the ability of the MLA to detect a specific biomarker such as amyloid, tau protein, or signs of Parkinson's disease in a multispectral image. If the quality threshold is a high value that needs to be reached or exceeded for the multispectral image to be considered suitable, then the stricter quality threshold has a higher value than the lenient quality threshold. If the quality threshold is a low value that should not be exceeded for the multispectral image to be considered suitable, then the stricter quality threshold has a lower value than the lenient quality threshold. As such, different quality thresholds may be applied in operation 440 according to the task at hand within the sequence 400.

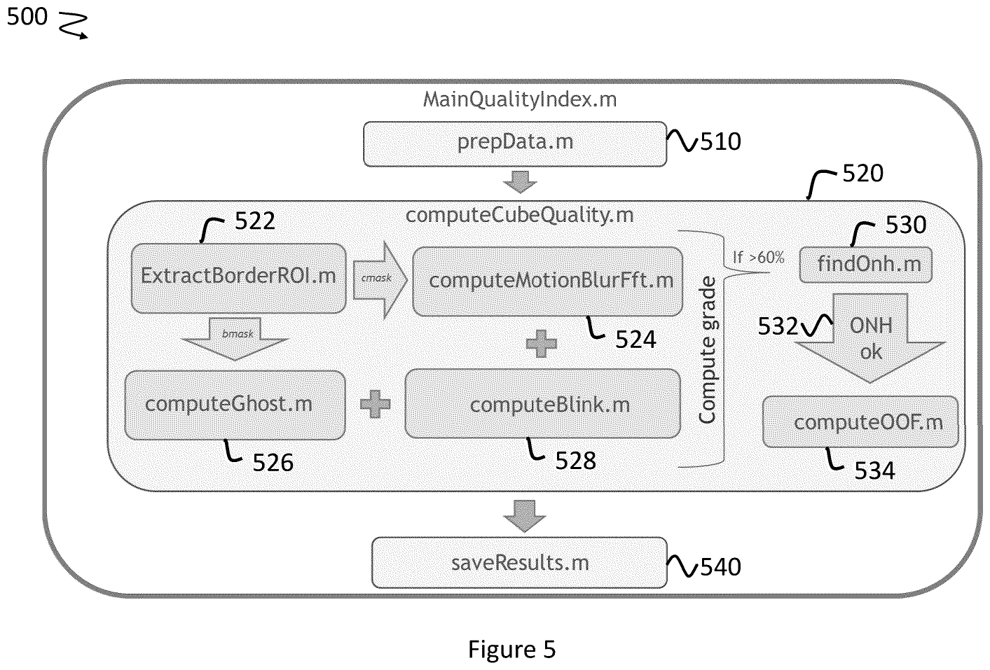

An example implementation of parts of operations 410 to 440 is illustrated in FIG. 5, which is a block diagram showing operations of a quality index calculation method 500. The order of the operations illustrated in non-limiting example of FIG. 5 may be used when attempting to detect features related to the presence of an amyloid biomarker in the retina of the subject. Data is prepared at operation 510, including acquiring the multispectral image of the retina (operation 410) and determining the first and second extents of the first and/or second artefacts (operations 420 and 430). In operation 510, the quality index is initialized as a first score value that represents a theoretical ideal image. The quality index is updated at operation 520 by evaluating the quality of the multispectral image of the retina (which may be described as a multispectral cube) and then stored as metadata for the multispectral image in a memory of the computer at operation 540.

Operation 520 includes several sub-operations 522-534, some of which may be optional. The execution order of the sub-operations 522-534 may differ from that shown on FIG. 5, at least for some of these sub-operations. A border of a region of interest (ROI) of the retina is extracted at sub-operation 522. A first mask (cmask) is applied on the ROI in order to compute a motion blur value for the ROI at sub-operation 524. A second mask (bmask) is applied on the ROI in order to compute a ghosting value for the ROI at sub-operation 526. The ROI is evaluated to determine whether the subject has blinked at the time of acquisition of the multispectral image in order to compute a blink value at sub-operation 528. The motion blur value, the ghosting value, and the blink value are combined with the first score value to compute an intermediate score value of the quality index. In an implementation as illustrated, each of the motion blur value, the ghosting value and the blink value is expressed as a percentage value that is deduced from an ideal value of 100%, which represents an ideal multispectral image of the retina. If the resulting intermediate score value of the quality index is less than 60%, the multispectral image of the retina is marked as ungradable. If the intermediate score value of the quality index is at least equal to 60%, the optical nerve head (ONH) position is searched at operation 530. This position is then verified at operation 532, in the manner described hereinbelow. If the operation 532 finds that the ONH position is not as expected, the multispectral image of the retina is marked as ungradable. Otherwise, an out-of-focus (OOF) value is calculated at operation 534 and used to modify the intermediate score value to provide a final value, or global score, of the quality index. In any event, the quality index is stored in the memory at operation 540, either as a numerical value, or as a grade indication (gradable, moderate, moderate_low, or ungradable, based on Table I above), or in both forms.

Details of the some of the sub-operations are provided in the next paragraphs.

### Blur Detection (Sub-Operation 524)

Sub-operation 524 detects a first artefact by assessing if there are any blurs in the multispectral image. Basically, it uses the concept of a Fourier transform to cut off high frequencies and see if this results in any major change in its values.

Sub-operation 524 first transforms a signal of the multispectral image in the Fourier domain for all its wavelengths. Doing so allows us to cut off high frequencies in the signal. Only a window of 200 by 200 pixels is kept at the center of the Fourier domain. Differences between consecutive images in the Fourier domain are calculated in the forward and the backward directions, resulting in 2 stacks of images (multispectral cubes). The sum of each value of each image transforms the multispectral cube into a vector. Each vector (forward and backward) is normalised using z-score technique.

The z-score of both of these vectors is then calculated independently for each of the values. To have a gold standard, a polynomial fit of second degree is computed with all of the values that are of 0.75 or less. A difference between each element of the vector and the polynomial fit vector is calculated for the forward and backward cases. To detect more accurately the blurred frame, the values for the forward and backward directions are multiplied to result in a single vector. Values of this vector can be used to detect blurred frames. A global threshold can be set experimentally to determine frames containing motion blur. Various thresholds for specific wavelength range can also be set depending on the required precision for a specific biomarker:

$$Frames 1 to 60 : Blur if value > 10$$

$$Frames 61 to 65 : Blur if value > 15$$

$$Frames 66 to 91 : Blur if value > 10$$

The above thresholds may be relaxed or strengthened depending on the actual MLA being used. At the end of sub-operation 524, a binary vector identifying where the blurs occurred in the multispectral image provides the motion blur value for each image or the cube.

### Ghost Detection (Sub-Operation 526)

Sub-operation 526 detects a second artefact by assessing if there are ghosts contained in the multispectral image. If any, the ghosts appear in the border of the regions of interest. So, using a mask (bmask) of the border of the ROI extracted by sub-operation 522, sub-operation 526 calculates differences between the frames (i.e. between the wavelengths) directly in the borders of the ROI. Normalisation using z-score is done in the third dimension (spectral dimension). The mean value and the standard deviation are calculated in the spectral dimension. For the mask of each frame, pixels whose values exceed 2 standard deviations from the mean are retained.

It was experimentally determined that if the number of pixels in the group is at least equivalent to 1% of the pixels contained in the mask, the frame is considered as having a ghost The function does this for each frame, and presence or not of ghost for each frames is saved in a binary vector in which value 1 indicates presence of a ghost. At the end of sub-operation 526 this binary vector contains information of presence (or not) of ghost for each frame.

### Blinks Detection (Sub-Operation 528)

Sub-operation 526 detects a third artefact by assessing if there are any blinks in the multispectral image. Blinks are fairly easy to detect since the effect on the multispectral images is drastic. Difference of consecutive full frames or portion of frames is calculated. Contrast of texture of the resulting image is computed. So, sub-operation 526 computes the contrast values on Gray Level Co-occurrence Matrix (specific types of textures) of the subtraction of two consecutive frames, covering the full wavelength range.

Sub-operation 526 processes three different spectral ranges differently, since the contrast in the multispectral image changes naturally between the various wavelengths: range 450-550 nm (frames 70 to 91), range 550-700 nm (frames 41 to 69) and finally range 700-900 nm (frames 1 to 40). For each of these ranges, a variation coefficient (VC) which represent the standard deviation divided by the mean, has been calculated for the contrast values. A blink in these positions will be detected if the VC reaches these thresholds:

$$Range 450 - 550 nm : Blink if VC > 45$$

$$Range 550 - 700 nm : Blink if VC > 40$$

$$Range 700 - 900 nm : Blink if VC > 20$$

Since changes are of lower magnitude for higher wavelengths, the thresholds are drastically reduced in order to permit the evaluation to have more sensitivity. After the algorithm have determined if there is a blink or not, it transfers the information to the rest of the programs. The blink value is obtained at the end of sub-operation 528.

### Optical Nerve Head (ONH) Position Detection (Sub-Operation 530)

The position of the ONH is detected at sub-operation 530. In particular, a position of the center is located. To this end, up to three different metrics of the multispectral image may be used, including the maximum variance, the min/max ratio, and the maximum value of a low pass filter. By combining the location of these three metrics, sub-operation 530 determines if the ONH is in a good range or not.

Sub-operation 530 may consider a single image from all the wavelengths of the multispectral image. Then, with this single image, the 3 metrics are computed to locate the position of the center of the ONH. The center of the ONH is found where the majority of these metrics are located, with a small margin of variation. From the literature, the most impactful of the three seems to be the maximum variance. Therefore, in case of important disagreement between the three ONH locations computed using the three metrics, the center of the ONH is simply determined using the maximum variance metric, instead of using the mean values of all metrics. With these calculations, the function will return if the ONH is correctly placed or not.

### Out-of-Focus (OOF) Detection (Sub-Operation 534)

Sub-operation 534 detects a fourth artefact by considering an eventual defocus in the multispectral images. In an implementation, sub-operation 534 may be omitted if results of the previous operations show that the quality index will be less than 80%. In this way, the operator will be able to see when the multispectral image is out of focus without having any of the other artefacts.

To this end, sub-operation 534 may evaluate the clearness of all blood vessels visible in the multispectral image of the retina. As such, a spectral region in the multispectral image (575-515 nm) is preprocessed to allow computing a frangi vesselness filter thereon. After this, a mask around all the blood vessels in the region is computed based on the intensity of the frangi filter. Finally, the square root of the variance of the values taken only in the region of the blood vessels is computed to give the out-of-focus (OOF) value.

### Quality Index

The quality index for the multispectral image is calculated based on results obtained for each artefact (Blur, blink, ONH position, ghost, out of focus), in sub-operations 524-534. A specific artefact might not have the same weight depending on which wavelength it was detected. For example, blurred frames detected in the wavelength range (600-700 nm) induce less variation in the global grade score compared to blurred frames in a lower wavelength range because this wavelength range contains less information for classification purposes. Moreover, some artefacts affect more than others the quality index. For example, if a blink is found, the quality index is affected at 100% rate, regardless at which wavelength it was found. Examples of the weights for each artefact in the context of amyloid biomarker are presented in Table 11:

TABLE II
Weight of each artefact depending on region

| | 450-600 nm | 600-700 nm | 700-900 nm |
|---|---|---|---|
| Blurs | 20% | 10% | 20% |
| Ghost | 10% | 10% | 10% |
| Blink | 100% | 100% | 100% |

Returning to FIG. 4b, the sequence 400 may end after operation 440, particularly if it is simply desired to evaluate the acquired multispectral image and store the result of the determination made at operation 440 in memory, for later use.

The sequence 400 may alternatively continue at operation 450, as shown on FIG. 4c. The computer may guide the operator by causing, at operation 450, the display of a pictogram 342, 344 or 346, the pictogram being associated with a color-code, the color-code being determined based on the quality index. As expressed hereinabove in the description of operation 350, when the quality index indicates that the multispectral image is ungradable, the pictogram 346 may further be associated with an indication of the first and/or the second artefacts associated with the first and/or second images of the captured multispectral image, respectively.

At operation 460, the computer considers the quality index obtained at operation 440 to select next events. If operation 460 finds that the multispectral image is suitable for training the MLA (the quality threshold being met), then operation 470 may follow, in which the MLA is trained based on the multispectral image of the retina, the trained MLA comprising a classification model being configured for detecting specific biomarkers and/or predicting medical conditions. Optionally, the multispectral image of the retina may, instead or in addition, be used by the computer for the detection of one or more medical conditions at operation 480. It is noted, however, that the quality threshold used to determine the suitability of the multispectral image operation 440 might be different depending on a desire to use the multispectral image for training of the MLA at operation 470, or for attempting to detect one or more medical conditions at operation 480.

If operation 460 finds that the multispectral image is not suitable for training the MLA, then operation 490 may follow.

At operation 490, the computer may generate instructions to acquire a replacement multispectral image of the retina. The instructions may comprise human-readable instructions to guide the operator with the acquisition of the replacement multispectral image of the retina. Operation (490 may include sub-operation 492, in which the computer iterates the generating instructions to acquire the replacement multispectral image of the retina until a new quality index for the replacement multispectral image at least fulfills the quality threshold.

They may be a desire to obtain a plurality of suitable multispectral images for training the MLA. In an implementation, the sequence 400 may be repeated until a predetermined number of successive multispectral images have been found suitable at operation 460 and used for training the MLA at operation 470. In the same or another implementation, the sequence 400 may be repeated until a combination of the quality indexes calculated at operation 430 for successive multispectral images at least fulfills a predetermined combined quality threshold.

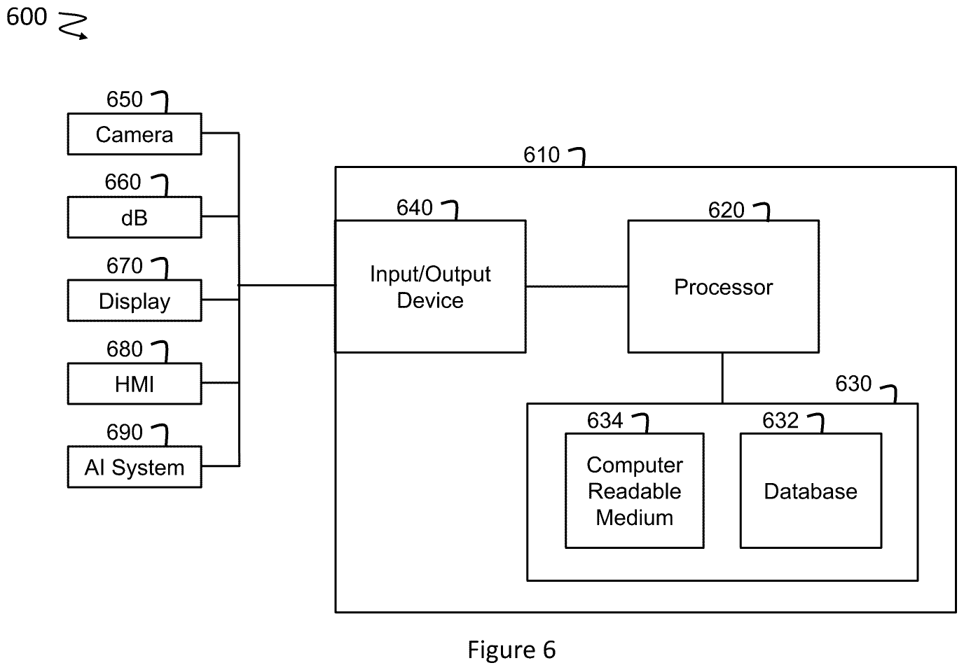

Each of the operations of the sequence 400 may be configured to be processed a computer having by one or more processors, the one or more processors being coupled to a memory device. For example, FIG. 6 is a block diagram of a computer-implemented system 600 for processing retinal images. On FIG. 6, the system 600 includes a computer 610 having a processor or a plurality of cooperating processors (represented as a processor 620 for simplicity), a memory device or a plurality of memory devices (represented as a memory device 630 for simplicity), an input device or a plurality of input/output devices (represented as an input/output device 640). The input/output device 640 may alternatively comprise a distinct input device and a distinct output device. The processor 620 is operatively connected to the memory device 630 and to the input/output device 640. The memory device 630 comprises a database 632 for storing, for example and without limitation, the multispectral image of the retina, parameters used by the routines executed at sub-operation 422, the quality threshold (or thresholds) applied at sub-operation 442 and/or at sub-operation 492, and the result of the suitability determination obtained at operation 440. The memory device 630 may also comprise a non-transitory computer-readable medium 634 for storing code instructions that are executable by the processor 620 for performing all or some of the operations of the sequence 400. In particular, the computer 600 may either implement the MLA and perform its training, or communicate the suitable multispectral images to an external AI system implementing the MLA.

The computer 610 may communicate, via the input/output device 640, with external devices including, for example and without limitation, the multispectral retinal camera 110 or a hyperspectral camera 650, an external database (dB) 660 storing multispectral images, a display device 670 for showing the human-readable instructions generated at operation 490 and/or the pictogram generated at operation at operation 450, a human-machine interface (HMI) 680 for receiving commands from the operator, and a trainable AI system 690 implementing the MLA.

While the description of FIGS. 4a-4c mainly relate to a computer-implemented method of processing retinal images, a computer-implemented method of processing other types of medical images is also contemplated. This method comprises accessing a multispectral image of a biological tissue, the multispectral image comprising a first image of the biological tissue associated with a first wavelength and a second image of the retina associated with a second wavelength, distinct from the first wavelength. A first extent of a first artefact associated with the first image is determined. A second extent of the first artefact or of a second artefact associated with the second image is also determined. A quality index based on the first extent of the first artefact and the second extent of the first or second artefact is computed. A determination is made, based on the quality index, that the multispectral image is suitable for detection of medical conditions.

This method may be used to determine whether a multispectral image of a retina is suitable for the detection of various medical conditions, for example by detecting the presence of amyloid plaques in the multispectral image of the retina. This method may also be used to evaluate a multispectral image of the skin of a subject for skin cancer detection. Use of a multispectral laparoscopic camera to obtain multispectral images of various internal body organs and tissues is also contemplated.

While the above-described implementations have been described and shown with reference to particular steps performed in a particular order, it will be understood that these steps may be combined, sub-divided, or re-ordered without departing from the teachings of the present technology. At least some of the steps may be executed in parallel or in series. Accordingly, the order and grouping of the steps is not a limitation of the present technology.

It should be expressly understood that not all technical effects mentioned herein need to be enjoyed in each and every implementation of the present technology.

As such, the computer-implemented methods, non-transitory computer-readable medium and computer-implemented systems implemented in accordance with some non-limiting implementations of the present technology can be represented as follows, presented in numbered clauses.

### Clauses

[Clause 1] A computer-implemented method of processing retinal images, the method comprising:
accessing a multispectral image of a retina, the multispectral image comprising a first image of the retina associated with a first wavelength and a second image of the retina associated with a second wavelength, the second wavelength being distinct from the first wavelength;
determining a first extent of a first artefact associated with the first image;
determining a second extent of the first artefact or of a second artefact associated with the second image;
computing a quality index based on the first extent of the first artefact and the second extent of the first or second artefact; and
determining, based on the quality index, whether the multispectral image is suitable for training a machine-learning algorithm (MLA).

[Clause 2] The method of clause 1, wherein accessing multispectral image comprises accessing a readable-computer medium comprising one or more files having been generated by a multispectral retinal camera.

[Clause 3] The method of clause 1 or 2, wherein determining, based on the quality index, whether the multispectral image is suitable for training the MLA comprises:
comparing the quality index with a quality threshold; and
if determination is made that the quality index does not at least fulfill the quality threshold, then determine that the multispectral image is not suitable for training the MLA.

[Clause 4] The method of clause 3, further comprising the step of.
if the multispectral image is deemed not to be suitable for the training of the MLA, generating instructions to acquire a replacement multispectral image of the retina.

[Clause 5] The method of clause 4, wherein the generating instructions to acquire the replacement multispectral image of the retina iterates until a new quality index for the replacement multispectral image at least fulfills the quality threshold, the quality threshold establishing that the replacement multispectral image is suitable for training the MLA.

[Clause 6] The method of clause 4 or 5, wherein the instructions comprise human-readable instructions to guide an operator with the acquisition of the at least one replacement multispectral image of the retina.

[Clause 7] The method of any one of clauses 3 to 6, wherein the quality threshold is associated with the first and/or the second artefacts.

[Clause 8] The method of any one of clauses 3 to 7, wherein the quality threshold is associated with a given biomarker.

[Clause 9] The method of any one of clauses 3 to 8, wherein the quality threshold is one of a first threshold usable for pretraining the MLA and a second threshold usable for further training of the MLA, the second threshold being stricter than the first threshold.

[Clause 10] The method of clause 9, wherein the further training of the MLA is used for fine tuning of the MLA on one or more specific tasks.

[Clause 11] The method of any one of clauses 1 to 10, further comprising displaying a pictogram, the pictogram being associated with a color-code, the color-code being determined based on the quality index.

[Clause 12] The method of any one of clauses 1 to 11, wherein multispectral image of the retina defines a hyperspectral cube, the hyperspectral cube being acquired by a hyperspectral camera.

[Clause 13] The method of any one of clauses 1 to 12, wherein the first and second wavelengths are adjacent within the multispectral image.

[Clause 14] The method of any one of clauses 1 to 13, wherein determining the first extent of the first artefact and determining the second extent of the first or second artefact are computed by executing at least one routine selected from:
a blur detection routine;
a ghost detection routine;
a blinks detection routine;
an optical nerve head (ONH) position detection routine;
a defocus detection routine; and
a combination thereof.

[Clause 15] The method of clause 14, wherein computing the quality index comprises:
initiating the quality index with a first numerical value; and
modifying the quality index to obtain a second numerical value of the quality index, the modifying of the quality index being based a result of executing at least one of the routines.

[Clause 16] The method of clause 14 or 15, wherein computing the quality index further comprises:
executing a plurality of the routines in an order selected according to a type of biomarker being sought in the multispectral image; and
associating distinct weights to each of the plurality of the routines, the weights being selected according to the type of biomarker being sought in the multispectral image.

[Clause 17] The method of anyone of clauses 1 to 16, wherein the quality index is associated with a gradeability score selected from gradable, moderate, moderate_low and ungradable.

[Clause 18] The method of anyone of clauses 1 to 17, further comprising training the MLA based on the multispectral image of the retina, the trained MLA comprising a classification model being configured for detecting specific biomarkers and/or predicting medical conditions.

[Clause 19] The method of any one of clauses 1 to 18, further comprising repeating the accessing of successive multispectral images and the determining whether the successive multispectral images are suitable for training the MLA until a predetermined number of suitable multispectral images have been acquired.

[Clause 20] The method of any one of clauses 1 to 19, further comprising repeating the accessing of successive multispectral images and the determining whether the successive multispectral images are suitable for training the MLA until a combination of the quality indexes for the successive multispectral images fulfills a predetermined combined quality threshold.

[Clause 21] A computer-implemented method of processing medical images, the method comprising:
accessing a multispectral image of a biological tissue, the multispectral image comprising a first image of the biological tissue associated with a first wavelength and a second image of the retina associated with a second wavelength, distinct from the first wavelength;
determining a first extent of a first artefact associated with the first image;
determining a second extent of the first artefact or of a second artefact associated with the second image;
computing a quality index based on the first extent of the first artefact and the second extent of the first or second artefact; and
determining, based on the quality index, that the multispectral image is suitable for detection of medical conditions.

[Clause 22] A non-transitory computer-readable medium storing computer-readable instructions that, when executed by a computer, cause the computer to perform the method of any one of clauses 1 to 21.

[Clause 23] A computer-implemented system configured to perform the method of any one of clauses 1 to 21.

Modifications and improvements to the above-described implementations of the present technology may become apparent to those skilled in the art. The foregoing description is intended to be exemplary rather than limiting. The scope of the present technology is therefore intended to be limited solely by the scope of the appended claims.

What is claimed is:

1. A computer-implemented method of processing retinal images, the method comprising:
accessing a multispectral image of a retina, the multispectral image comprising a first image of the retina associated with a first wavelength and a second image of the retina associated with a second wavelength, the second wavelength being distinct from the first wavelength;
determining a first extent of a first artefact associated with the first image;
determining a second extent of the first artefact or of a second artefact associated with the second image;
computing a quality index based on the first extent of the first artefact and the second extent of the first or second artefact; and
determining, based on the quality index, whether the multispectral image is suitable for training a machine-learning algorithm (MLA).

2. The method of claim 1, wherein accessing multispectral image comprises accessing a readable-computer medium comprising one or more files having been generated by a multispectral retinal camera.

3. The method of claim 1, wherein determining, based on the quality index, whether the multispectral image is suitable for training the MLA comprises:
comparing the quality index with a quality threshold; and
if determination is made that the quality index does not at least fulfill the quality threshold, then determine that the multispectral image is not suitable for training the MLA.

4. The method of claim 3, further comprising the step of:
if the multispectral image is deemed not to be suitable for the training of the MLA, generating instructions to acquire a replacement multispectral image of the retina.

5. The method of claim 4, wherein the generating instructions to acquire the replacement multispectral image of the retina iterates until a new quality index for the replacement multispectral image at least fulfills the quality threshold, the quality threshold establishing that the replacement multispectral image is suitable for training the MLA.

6. The method of claim 4, wherein the instructions comprise human-readable instructions to guide an operator with the acquisition of the at least one replacement multispectral image of the retina.

7. The method of claim 3, wherein the quality threshold is associated with the first and/or the second artefacts.

8. The method of claim 3, wherein the quality threshold is associated with a given biomarker.

9. The method of claim 3, wherein the quality threshold is one of a first threshold usable for pretraining the MLA and a second threshold usable for further training of the MLA, the second threshold being stricter than the first threshold.

10. The method of claim 9, wherein the further training of the MLA is used for fine tuning of the MLA on one or more specific tasks.

11. The method of claim 1, further comprising displaying a pictogram, the pictogram being associated with a color-code, the color-code being determined based on the quality index.

12. The method of claim 1, wherein multispectral image of the retina defines a hyperspectral cube, the hyperspectral cube being acquired by a hyperspectral camera.

13. The method of claim 1, wherein the first and second wavelengths are adjacent within the multispectral image.

14. The method of claim 1, wherein determining the first extent of the first artefact and determining the second extent of the first or second artefact are computed by executing at least one routine selected from:
a blur detection routine;
a ghost detection routine;
a blinks detection routine;
an optical nerve head (ONH) position detection routine;
a defocus detection routine; and
a combination thereof.

15. The method of claim 14, wherein computing the quality index comprises:
initiating the quality index with a first numerical value; and
modifying the quality index to obtain a second numerical value of the quality index, the modifying of the quality index being based a result of executing at least one of the routines.

16. The method of claim 1, further comprising training the MLA based on the multispectral image of the retina, the trained MLA comprising a classification model being configured for detecting specific biomarkers and/or predicting medical conditions.

17. The method of claim 1, further comprising repeating the accessing of successive multispectral images and the determining whether the successive multispectral images are suitable for training the MLA until a predetermined number of suitable multispectral images have been acquired.

18. The method of claim 1, further comprising repeating the accessing of successive multispectral images and the determining whether the successive multispectral images are suitable for training the MLA until a combination of the quality indexes for the successive multispectral images fulfills a predetermined combined quality threshold.

19. A computer-implemented method of processing medical images, the method comprising:
accessing a multispectral image of a biological tissue, the multispectral image comprising a first image of the biological tissue associated with a first wavelength and a second image of the retina associated with a second wavelength, distinct from the first wavelength;
determining a first extent of a first artefact associated with the first image;
determining a second extent of the first artefact or of a second artefact associated with the second image;
computing a quality index based on the first extent of the first artefact and the second extent of the first or second artefact; and
determining, based on the quality index, that the multispectral image is suitable for detection of medical conditions.

20. A non-transitory computer-readable medium storing computer-readable instructions that, when executed by a computer, cause the computer to perform the method of:
accessing a multispectral image of a retina, the multispectral image comprising a first image of the retina associated with a first wavelength and a second image of the retina associated with a second wavelength, the second wavelength being distinct from the first wavelength;
determining a first extent of a first artefact associated with the first image;
determining a second extent of the first artefact or of a second artefact associated with the second image;
computing a quality index based on the first extent of the first artefact and the second extent of the first or second artefact; and
determining, based on the quality index, whether the multispectral image is suitable for training a machine-learning algorithm (MLA).
